(19) United States
(12) Patent Application Publication
(10) Pub. No.: US 2024/0081842 A1
(43) Pub. Date: Mar. 14, 2024

(54) BONE CUTTING GUIDE TOOL ASSEMBLY FOR ARTIFICIAL JOINT SURGERY

(71) Applicant: CONNECTEVE CO., Ltd., Seoul (KR)

(72) Inventor: Du Hyun RO, Seoul (KR)

(73) Assignee: CONNECTEVE CO., Ltd., Seoul (KR)

(21) Appl. No.: 18/213,271

(22) Filed: Jun. 23, 2023

(30) Foreign Application Priority Data
Sep. 13, 2022 (KR) 10-2022-0114728

(51) Int. Cl. A61B 17/17; A61B 17/04; A61B 17/88

(52) U.S. Cl. CPC A61B 17/1717; A61B 17/0401; A61B 17/8897; A61B 2017/0409

(57) ABSTRACT

Provided are a bone cutting guide tool assembly for artificial joint surgery. The assembly comprises a base unit having a through hole, a guide rod inserted into the through hole and being inserted into a bone through a hole drilled at an end of the bone, a positioning unit having a plurality of holes for driving a pin into the bone in contact with the bone, a connection unit mechanically connecting the base unit and the positioning unit so that the base unit and the positioning unit is enabled to slide vertically with each other, and a guide unit having a pin insertion hole to be inserted into the pin and seated on the bone.

### A. TECHNICAL FIELD

The present disclosure relates to cutting a bone during artificial joint surgery, and more specifically, to a bone cutting guide tool assembly for artificial joint surgery that guides a cutter of a bone cutting device in a direction corresponding to the shape of the end of the bone, so that a cutting section of the bone forms an angle parallel to a slope of the end curvature of the bone.

### B. DESCRIPTION OF THE RELATED ART

As human lifespan has greatly increased compared to the past, cartilage constituting each joint that plays an important role in human body movement is completely worn out before the end of human lifespan, and degenerative arthritis occurs frequently, and this causes a significant decrease in quality of life in the process of spending the rest of human life.

Recently, in order to medically solve these problems, artificial joint surgery that replaces the original joint with metal, plastic, or ceramic has been actively performed, and furthermore, research activities on surgical techniques for better surgical results.

In general, artificial joint surgery can be applied to all joints, but most of them are performed on the hip joint and knee joint. In this case, the surface of the bone forming the joint is finely trimmed, thin special metal is covered, and plastic (polyethylene insert) is placed between the femur and tibia. By allowing it to act as a cartilage, it allows the hip and knee joints to move flexibly and normally.

In order to do this, the end of the bone constituting the joint is cut by a predetermined length, and a process of inserting a replacement artificial joint having a shape corresponding to the cut portion is performed.

However, in the past, the process of cutting a bone by a predetermined length often relied on, the operator's eye measurement or his/her experience using surgical instruments, so a situation in which the replacement joint did not fit accurately often occurred, which causes a significant drop in the patient's satisfaction with surgery.

In order to solve this problem, a device for guiding a cutter that directly cuts bone in the process of cutting bone has been devised, but the conventionally developed guide devices mostly set an approximate cutting position through. an intramedullary femoral implant device. Because of this, there is a problem that some part of the error occurs. Therefore, a method for solving these problems is required.

### SUMMARY OF THE DISCLOSURE

In one aspect of the present disclosure, a guide unit for bone cutting guide tool assembly comprises a body having at least one guide slit; and an anchor configured to extend from a side of the body, having a first pin insertion hole and a second pin insertion hole with elongated shape therein.

The anchor may be configured to further have a third pin insertion hole and the fourth pin insertion hole between the first pin insertion hole and the second pin insertion hole, and the third pin insertion hole is formed in the anchor so as to be slanted with respect to the fourth pin insertion hole.

A shape of the first pin insertion hole may be subsequently same to a shape of the third pin insertion hole and the fourth pin insertion hole.

A straight line connecting the first pin insertion hole and the second pin insertion hole may be parallel to a straight line extended from length of the guide slit.

In another aspect of the present disclosure, a bone cutting guide tool assembly comprises a base unit having a through hole; a guide rod inserted into the through hole and being inserted into a bone through a hole drilled at an end of the bone; a positioning unit having a plurality of holes for driving a pin into the bone in contact with the bone; a connection unit mechanically connecting the base unit and the positioning unit so that the base unit and the positioning unit is enabled to slide vertically with each other; and a guide unit having a pin insertion hole to be inserted into the pin and seated on the bone.

The base unit may include a rod fixing part having the through hole and a movement guide part having a channel to slidably engage with the connection unit.

The positioning unit may include a reference designation part having the plurality of holes and a moving part having a protrusion on a side of the moving part to slidably engage with the connection unit.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 is a diagram illustrating a bone cutting guide tool assembly for artificial joint surgery according to embodiments of the present disclosure.

FIG. 2 is a diagram illustrating a guide rod of the bone cutting guide tool assembly for artificial joint surgery according to embodiments of the present disclosure.

FIG. 3 is a diagram illustrating a base unit, a positioning unit, and a connection unit of the bone cutting guide tool assembly for artificial joint surgery according to embodiments of the present disclosure.

FIGS. 4 and 5 are diagrams illustrating a guide unit of the bone cutting guide tool assembly viewed from one side, respectively according to embodiments of the present disclosure.

FIGS. 6 to 15 are diagrams sequentially illustrating artificial joint surgery procedures using the bone cutting guide tool assembly according to embodiments of the present disclosure.

### DETAILED DESCRIPTION OF PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, or a device.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof.

It shall also be noted that the terms “coupled,” “connected,” “linked,” or “communicatively coupled” shall be understood to include direct connections, indirect connections through one or more intermediary devices.

Furthermore, one skilled in the art shall recognize: (1) that certain steps may optionally be performed; (2) that steps may not be limited to the specific order set forth herein; and (3) that certain steps may be performed in different orders, including being done contemporaneously.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

The terms “comprise/include” used throughout the description and the claims and modifications thereof are not intended to exclude other technical features, additions, components, or operations.

Unless the context clearly indicates otherwise, the singular forms “a,” “an,” and “the” are intended to include the plural forms as well. Also, when description related to a known configuration or function is deemed to render the present disclosure ambiguous, the corresponding description is omitted.

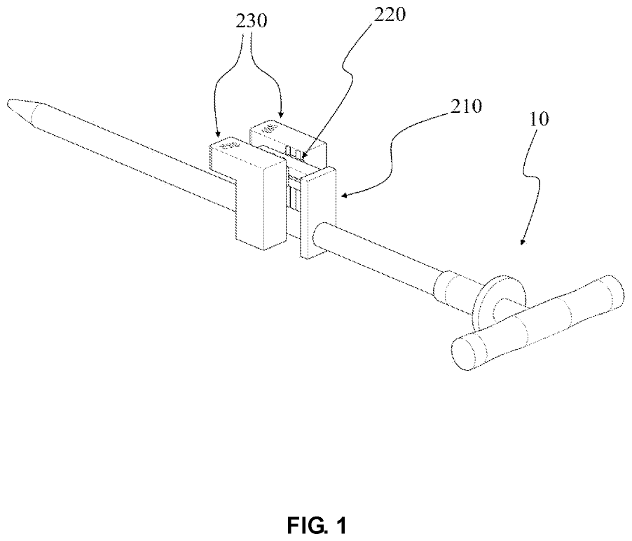

FIGS. 1 to 5 are a diagram illustrating a bone cutting guide tool assembly for artificial joint surgery according to embodiments of the present disclosure.

As depicted, the bone cutting guide tool assembly may include a guide rod 10, a base unit 210, a positioning unit 230, a connection unit 220 and a guide unit 100. Hereinafter, through FIGS. 1 to 5, the each of the components of the bone cutting guide tool assembly will be described according to the order used in the artificial joint surgery.

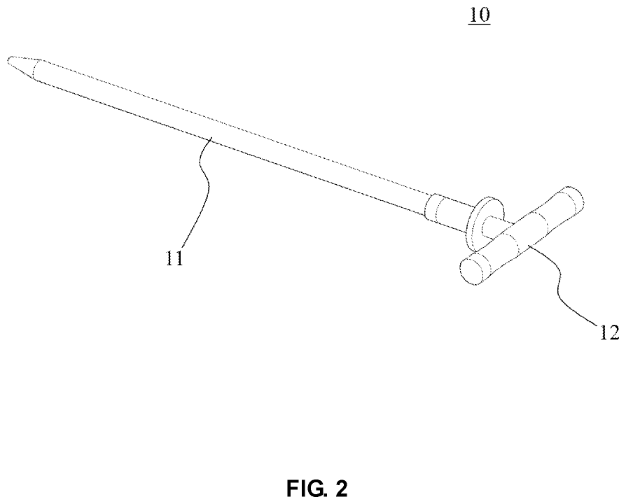

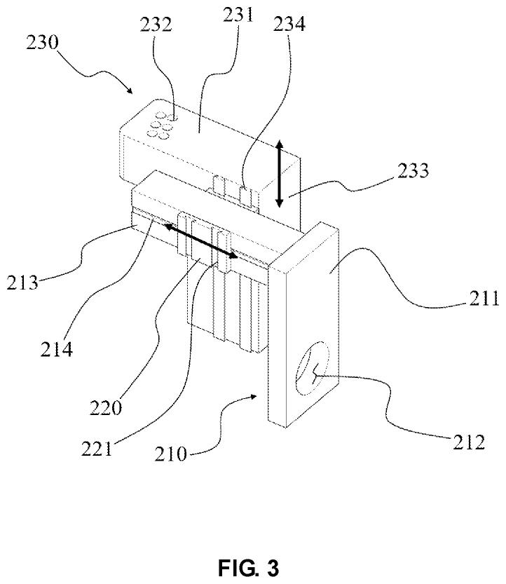

FIG. 1 is a diagram illustrating a guide rod 10, a base unit 210, a positioning unit 230, and a connection unit 220 that are combined in the bone cutting guide tool assembly for artificial joint surgery according to an embodiment of the present disclosure, FIG. 2 is a diagram illustrating a guide rod 10 in the bone cutting guide tool assembly for artificial joint surgery according to an embodiment of the present disclosure, and FIG. 3 is a partial exploded view illustrating a base unit 210, a connection unit 220 and a positioning unit 230 in the bone cutting guide tool assembly for artificial joint surgery according to an embodiment of the present disclosure.

Each of the components shown in FIGS. 1 to 3 are components previously used to determine the installation position of the guide unit 100.

In the following description, terms indicating directions are based on the directions indicated in the drawings. However, the term is only set for convenience of explanation and is not intended to limit the rights of the present invention.

As depicted in FIG. 2, the guide rod 10 may be formed by T-shaped structure and be provided to be inserted into a bone through a hole drilled at the end of the bone to be operated. In embodiments, the guide rod 10 may include a rod part 11 which is formed long in the longitudinal direction and a grip part 12 which is provided at the end of the rod part so that the operator can grip the grip part 12. In embodiments, a distal part of the rod part 11 may be introduced into a drilling hole formed in the bone, and the distal part of the rod part 11 may be formed in a shape in which the cross-sectional area is gradually reduced to facilitate insertion into the hole of the bone. In embodiments, the grip part 12 may be provided at a proximal part of the rod part 11 and may be vertically connected to the rod part so that the operator can easily grip it.

As depicted in FIG. 3, the base unit 210 may include a rod fixing part 211 and a movement guide part 213 having a predetermined length, extending in a vertical direction from a side of the rod fixing part 211. In embodiments, the rod fixing part 211 may have a hole 212 which the rod part 11 of the guide rod 10 passes through and the movement guide part 213 may have a channel 214 formed in a longitudinal direction of the movement guide part 213.

Although the positioning unit 230 is shown by a single unit, it is formed by a pair. In embodiments, each positioning unit 230 may be mounted on both sides of the movement guide part 213 of the base unit 210. In embodiments, each positioning unit 230 may be formed to be movable in a forward and backward direction of the positioning unit 210 along the channel 214 formed in the movement guide part 213 or in an up and down direction of the positioning unit 210 along a groove 221 formed in the connection unit 220 within a set range with respect to the movement guide part 213 of the base unit 210.

Accordingly, each positioning unit 230 is able to determine the installation position of the reference pin for seating the guide unit 100 illustrated in FIG. 5 on one side of the bone while being moved to meet the end of bone.

In embodiments, each positioning unit 230 may have a moving part 233 and a reference designation part 231. In embodiments, the moving part 233 may have a protrusion 234 on one side of the moving part 233 and a front surface of the moving part 233 may be formed as a reference plane in contact with the end of the bone. In embodiments, the reference designation part 231 may be extend in a vertical direction of one side of the moving part 233 to be in parallel to a longitudinal direction of the movement guide part 213. As such, the installation position of the reference pin may be determined on an arbitrary part of the reference designation part 231 in a state where the moving part 233 is moved so that the reference plane comes into contact with the end of the bone. In this case, the reference designation part 231 may have a plurality of pin installation holes 232 to enable the reference pins to insert therein. In embodiments, the plurality of pin installation holes 232 may be disposed at a different position with a given distance.

Meanwhile, the connection unit 220 may be provided between the movement guide part 213 of the base unit 210 and the moving part 233 of the positioning unit 230. In embodiments, the connection unit 220 may be mechanically or structurally connected to the movement guide part 213 so as to be able to slide along the channel 214 of the movement guide part 213. Also, the connection unit 220 may be mechanically or structurally connected to the moving part 233 so that the protrusion 234 of the moving part 233 is capable of sliding along the groove 221 of the connection unit 220. Thus, the base unit 210 and the positioning unit 230 may be enabled to slide vertically with each other. In embodiments, the moving part 233 may have at least protrusion 234 and the groove 221 may be formed to correspond to the protrusion 234.

In embodiments, the channel 214 may be formed in the moving guide unit 213 in a longitudinal direction of the moving guide unit 213 so that the connection unit 220 can move along the channel 214 of the moving guide unit 213 and the groove 221 may be formed in the connection unit 220 so that the moving part 233 with the protrusion 233 can move along the groove 221 of the connection unit 220.

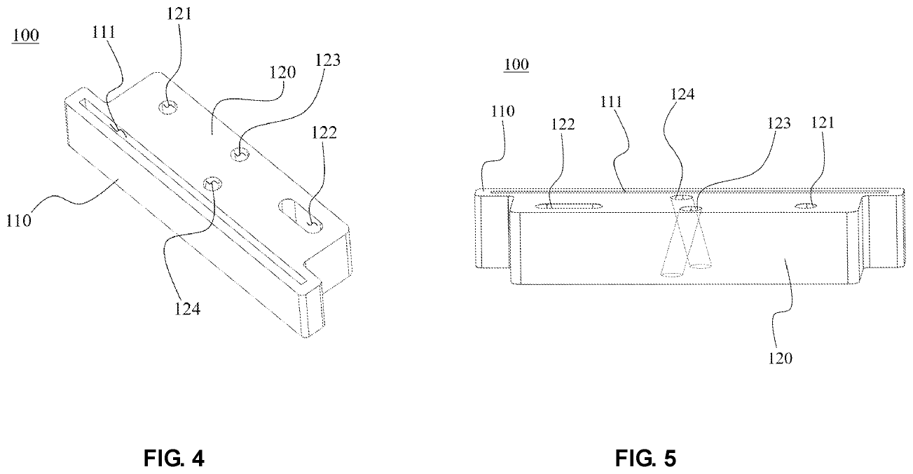

FIGS. 4 and 5 are views illustrating a guide unit of a bone cutting guide tool assembly for artificial joint surgery according to an embodiment of the present disclosure.

As depicted in FIGS. 4 and 5, the guide unit 100 may include a body 110 and an anchor 120 extending from a side of the body 110. In embodiments, the body 110 may have a guide slit 111 that is formed in a longitudinal direction of the body 110. The body 110 may have a plurality of guide slits. In this case, the plurality of the guide slits may be disposed on the body 110 parallel to each other. In embodiments, the anchor 120 may have a first pin insertion hole 121, a second pin insertion hole 122 having an elongated shape, a third pin insertion hole 123 and a fourth pin insertion hole 124 therein so that the anchor 120 is fixed to a pin put into a bone. In embodiments, the first pin insertion hole 121 to the fourth pin insertion hole 124 may be disposed at a different position of the anchor 120.

The first pin insertion hole 121 and the second pin insertion hole 122 may be fixed by being inserted respectively into a pair of reference pins put in the bone through the pin installation hole 232 of each of the positioning unit 230 shown in FIG. 3. In embodiments, the first pin insertion hole 121 and the second pin insertion hole 122 may be placed apart from each other on both edge portions of the anchor 120. Thus, a straight line connecting the first pin insertion hole 121 and the second pin insertion hole 122 may be parallel to a straight line extended from length of the guide slit 111.

Meanwhile, the installation position of the reference pins may vary in according with a shape of the bone for every surgical procedure. Accordingly, the second pin insertion hole 122 may be formed in the elongated or oblong shape relative to the first pin insertion hole 121, so as to secure an insertion margin for the second reference pin put on the other side of the bone in a state where the first pin insertion hole 121 is inserted into the first reference pin put on one side of the bone.

In addition, the third pin insertion hole 123 and the fourth pin insertion hole 124 may be space apart from each other with a given distance on the center part between the first pin insertion hole 121 and the second pin insertion hole 122 so that anchoring pins for fixing the guide unit 100 itself to one side of the bone can be inserted. In embodiments, the reference pin is for designating the installation position of the guide unit 100 on the bone, and the anchoring pin is for tightly fixing the guide unit 100 to the corresponding installation position of the bone.

In embodiments, the third pin insertion hole 123 and the fourth pin insertion hole 124 may be formed in a shape inclined by a predetermined angle with respect to a virtual vertical line of the surface of the anchor, which is to further increase the fixing force of the guide unit 100 by allowing anchoring pins inserted into the third pin insertion hole 123 and the fourth pin insertion hole 124 to be installed at an inclined angle with respect to the bone. In embodiments, the third pin insertion hole 123 and the fourth pin insertion hole 124 may be formed in the form of slanting in opposite directions to maximize the fixing force of the guide unit 100.

Hereinafter, the process of performing artificial joint surgery through each component of the present embodiment will be described.

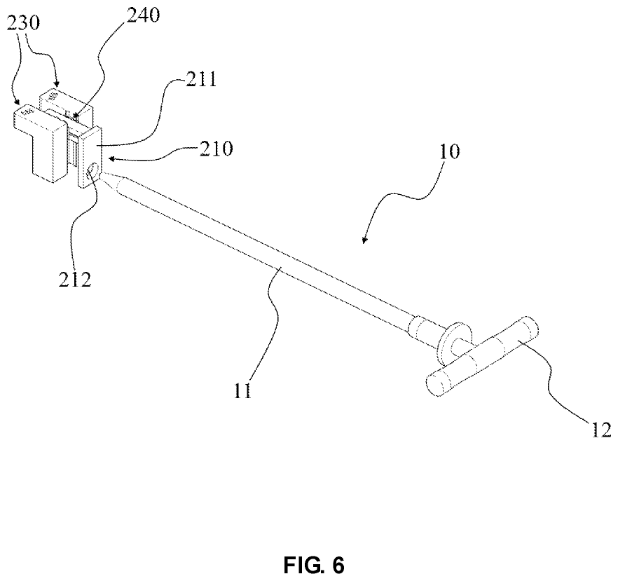

FIGS. 6 to 15 are diagrams sequentially illustrating artificial joint surgery procedures using the bone cutting guide tool assembly according to embodiments of the present disclosure.

As depicted in FIG. 6, the guide rod 10, the base unit 210, the positioning unit 230 and the connection unit 240 are prepared. Among this things, the base unit 210, the positioning unit 230 and the connection unit 240 are pre-combined to each other, and then the guide rod 10 is structurally combined to the base unit 210 by allowing the rod part 11 of the guide rod 10 inserted into the hole 212 formed in the rod fixing part 211 of the base unit 210.

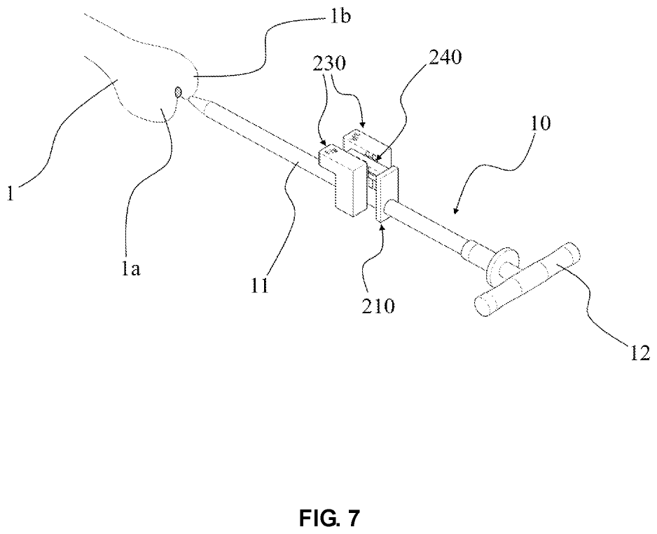

FIG. 7 illustrates a state in which the guide rod, the base unit, the positioning unit 230 and the connection unit 240 are combined to each other. In general, since the end of the bone 1 is formed to be curved, the maximum protruding part may be different depending on patients. The end of the bone 1 of the femoral region shown in the present embodiment forms a curve in which both side parts (1a, 1b) protrude from the center, and among them, the first end 1a was set as having a more protruding state compared to the other second end 1b.

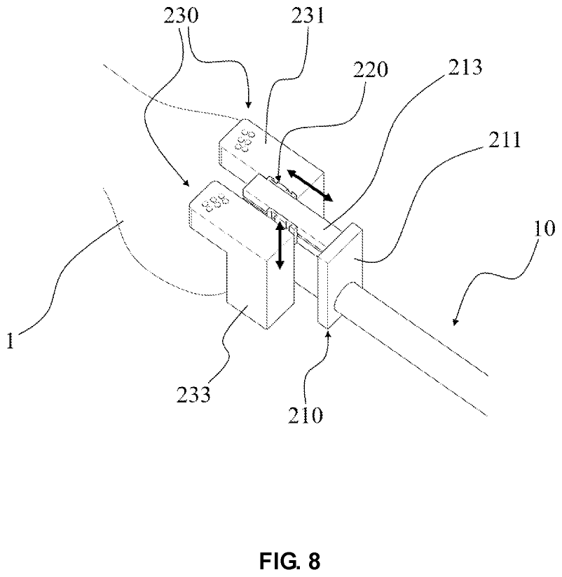

As depicted in FIG. 7, after forming a drilling hole through a tool such as a drill at the end of the bone to be operated on, the rod part 11 of the guide rod 10 into the drilling hole to the bone is inserted and is drawn into the inside of the bone 1. At this time, the rod part 11 may be inserted inside the bone 1 by an operator so that a reference plane of the moving part 233 provided in the positioning unit 230 reaches to a protruding part formed at the end of the bone 1. In this state, as depicted in FIG. 8, the operator may move each the positioning unit 230 in the forward and backward directions and up and down directions based on the base unit 210, and the reference plane formed on moving part 233 is in contact with the protruding portion formed at the end of the bone 1.

In this process, each positioning unit 230 may be independently moved to different positions according to the bending of the end of the bone 1. In embodiments, the reference plane formed on the moving part 233 of the positioning unit 230 located on the left is in contact with the first end 1a of the bone 1, and the reference plane formed on the moving part 233 of the positioning unit 230 located on the right is in contact with the second end 1b of the bone 1.

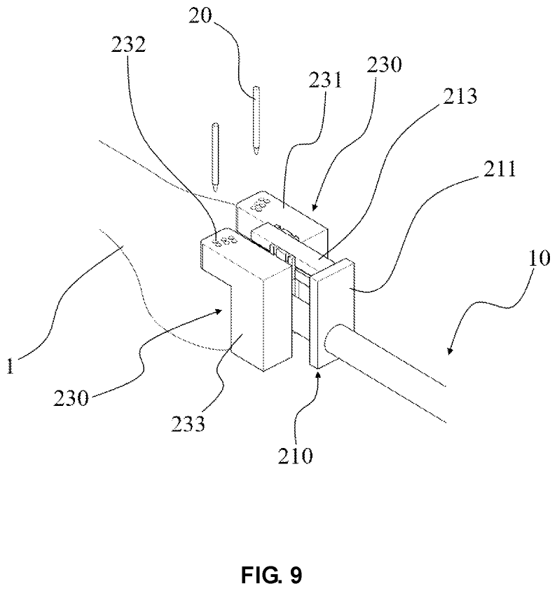

After setting the positions of each the positioning unit 230 in the above way, as depicted in FIG. 9, a process of inserting the reference pin 20 into an arbitrary pin installation hole among a plurality of pin installation holes 232 formed in each reference designation part 231 is performed thereby the reference pin 20 is driven into the bone 1.

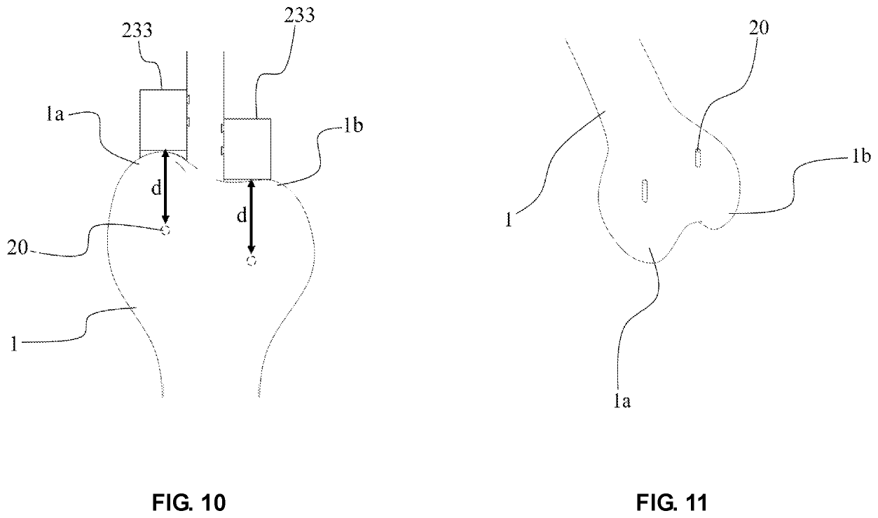

FIG. 10 illustrates a view from the bottom of the bone in the process of putting the reference pin into the bone.

As depicted in FIG. 10, even though each the moving part 233 of the positioning unit is located at different positions according to the end curve of the bone 1, each the reference pin 20 may be driven into the bone 1 at the same distance (d) from the first end 1a or the second end 1b of the bone 1 of the moving part 233. Also, depending on where the reference pin 20 is inserted into the pin installation hole, each the reference pin 20 may be driven into the bone 1 at the different distance (d) from the first end 1a or the second end 1b of the bone 1 of the moving part 233.

After that, in a state where each the reference pin 20 is fixed to the upper part of the bone 1, as depicted in FIG. 11, the guide rod 10, the base unit 210, the positioning unit 230 and the connection unit 220 are removed from the bone 1. Thereby, only the reference pin 20 remains on the bone 1.

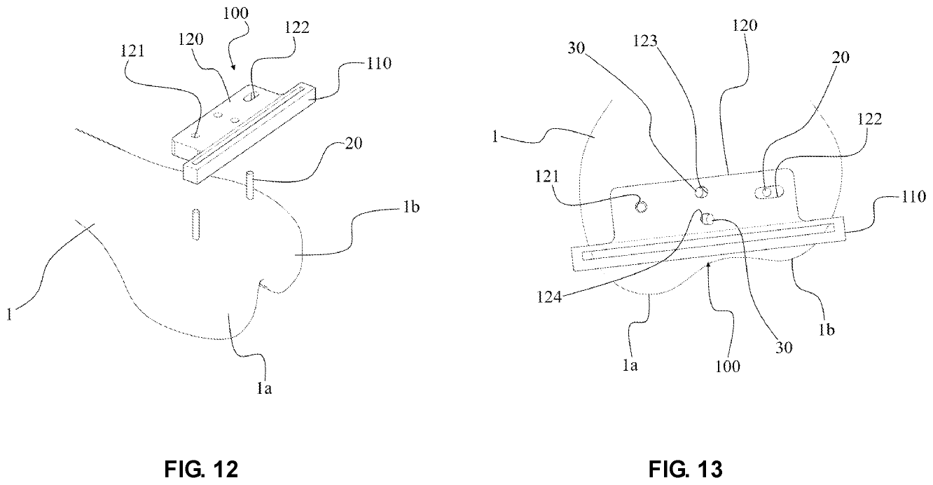

Next, as depicted in FIG. 12, a guide unit 100 is prepared, and each the reference pin 20 fixed to the bone 1 may be inserted into the first pin insertion hole 121 and the second pin insertion hole 122 that is formed on the anchor 120 of the guide unit 100. As described above, since the second pin insertion hole 122 is formed in the elongated shape, in a state in which one reference pin 20 is inserted into the first pin insertion hole 121, the second pin insertion hole 122 can secure an insertion margin range for the other reference pin 20.

According to the above process, the position of the guide unit 100 can be set on the bone 1 through the reference pin 20.

Then, as depicted in FIG. 13, as the anchoring pin 30 is inserted into the third pin insertion hole 123 and the fourth pin insertion hole 124 formed in the anchor 120 of the guide unit 100, the guide unit 100 and the bone 1 is tightly combined to each other. As described above, since the third pin insertion hole 123 and the fourth pin insertion hole 124 may be formed in the form tilting in opposite directions to each other with a predetermined angle with respect to a virtual vertical line, the anchoring pin 30 may be installed at an inclined angle with respect to a bone 1 and be further increase the fixing force of the guide unit 100.

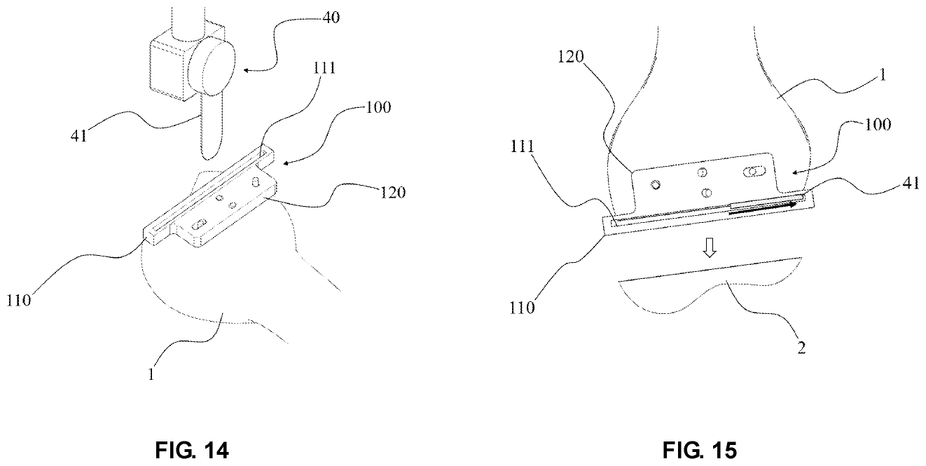

And then, as depicted in FIGS. 14 and 15, a bone cutting device 40 including a cutter 41 is prepared, the cutter 41 of the bone cutting device 40 is inserted into the guide slit 111 of the guide unit 100, and then the bone 1 is cut along the guide slit 111 by the cutter 41. Herein, since a longitudinal direction of the guide slit 111 is parallel to the straight line connecting each the reference pin, the cutting direction of the bone 1 also becomes the longitudinal direction of the guide slit 111. Accordingly, the piece of cut bone is removed from the bone 1, and then a process of mounting an alternative joint mechanism for an artificial joint to the cutting position of the bone 1 body may be performed.

In the description, numerous details are set forth for purposes of explanation in order to provide a thorough understanding of the present invention. However, it will be apparent to one skilled in the art that not all of these specific details are required in order to practice the present invention.

Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. A guide unit for bone cutting guide tool assembly, comprising:
a body having at least one guide slit; and
an anchor configured to extend from a side of the body, having a first pin insertion hole and a second pin insertion hole with elongated shape therein.

2. The guide unit of claim 1,
wherein the anchor is configured to further have a third pin insertion hole and the fourth pin insertion hole between the first pin insertion hole and the second pin insertion hole, and
wherein the third pin insertion hole is formed in the anchor so as to be slanted with respect to the fourth pin insertion hole.

3. The guide unit of claim 2,
wherein a shape of the first pin insertion hole is subsequently same to a shape of the third pin insertion hole and the fourth pin insertion hole.

4. The guide unit of claim 2,
wherein a straight line connecting the first pin insertion hole and the second pin insertion hole is parallel to a straight line extended from length of the guide slit.

5. A bone cutting guide tool assembly for artificial joint surgery, comprising:
a base unit having a through hole;
a guide rod inserted into the through hole and being inserted into a bone through a hole drilled at an end of the bone;
a positioning unit having a plurality of holes for driving a pin into the bone in contact with the bone;
a connection unit mechanically connecting the base unit and the positioning unit so that the base unit and the positioning unit is enabled to slide vertically with each other; and
a guide unit having a pin insertion hole to be inserted into the pin and seated on the bone.

6. The bone cutting guide tool assembly of the claim 5,
wherein the base unit includes a rod fixing part having the through hole and a movement guide part having a channel to slidably engage with the connection unit.

7. The bone cutting guide tool assembly of claim 5,
wherein the positioning unit includes a reference designation part having the plurality of holes and a moving part having a protrusion on a side of the moving part to slidably engage with the connection unit.

8. The bone cutting guide tool assembly of claim 5,
wherein the guide unit includes a body having at least one guide slit and an anchor configured to extend from a side of the body, and
wherein the pin insertion hole includes a first pin insertion hole and a second pin insertion hole with elongated shape.

9. The bone cutting guide tool assembly of claim 8,
wherein the anchor is configured to further have a third pin insertion hole and the fourth pin insertion hole between the first pin insertion hole and the second pin insertion hole, and
wherein the third pin insertion hole is formed in the anchor so as to be slanted with respect to the fourth pin insertion hole.
